(19) United States
(12) Patent Application Publication
(10) Pub. No.: US 2006/0058777 A1
(43) Pub. Date: Mar. 16, 2006

(54) Urinary catheter device with a pharmaceutically active composition

(76) Inventor: Pia Norup Nielsen, Rungsted Kyst (DK)

Correspondence Address: JACOBSON HOLMAN PLLC, 400 SEVENTH STREET N.W., SUITE 600, WASHINGTON, DC, 20004, US

(21) Appl. No.: 10/538,832

(22) PCT Filed: Dec. 11, 2003
(86) PCT No.: PCT/DK03/00853

§ 371 Date: Jun. 13, 2005

Related U.S. Application Data
(63) Continuation-in-part of application No. 60/434,661, filed on Dec. 20, 2002.

(30) Foreign Application Priority Data
Dec. 11, 2002 (DK) PA 2002 01899

(51) Int. Cl. A61F 5/44

(52) U.S. Cl. 604/544; 604/329

(57) ABSTRACT

The invention relates to a device for urinary catheterisation comprising a catheter element and a pharmaceutically active composition containing a hormone, an efferent blocking agent, an afferent blocking agent and/or a sympathomimetic agent, the catheter element being adapted to deliver the pharmaceutically active composition in the lower urinary tract system during catheterisation. The invention also relates to the use of said pharmaceutically active composition for the manufacture of a device for the treatment, alleviation or prophylaxis of incontinence in a human and to a method of treating a human suffering from or being susceptible to incontinence. The invention further relates to a device for urinary catheterisation comprising a discrete unit dose comprising a pharmaceutically active composition and a catheter element adapted to shed said pharmaceutically active composition in the lower urinary tract system during catheterisation.

### FIELD OF THE INVENTION

The present invention relates to a urinary catheter with a pharmaceutically active composition.

### BACKGROUND OF THE INVENTION

Urinary incontinence is involuntary loss of urine from the bladder and affects millions of people worldwide. It can be caused by a great variety of conditions, including weak pelvic floor and sphincter muscles, estrogen deficiency, traumatic lesions of the urinary system or lesions of peripheral nerves innervating the bladder. Furthermore, spinal cord injury or central nervous diseases or lesions can cause urinary incontinence.

In some cases, urinary incontinence is seen in combination with overactive bladder and difficulties with complete emptying of the bladder. Intermittent catheterisation is the preferred method of bladder emptying, in the case of over active bladder. Often this method is combined with medical treatment to relax the bladder musculature and increase bladder capacity. In general, systemic drug treatment affects the whole body and has a high risk of side-effects. Local treatment reduces the risk of side-effects, and in some cases increases efficacy.

A special problem applies specifically to women using catheterisation as the method of emptying the bladder. In particular menopausal and postmenopausal women often experience symptoms caused by the estrogen deficiency, including thinning of the urethral mucosa and vaginal mucosa. The uro-genital oestrogen deficiency syndrome includes local urogenital symptoms, appearing in 25-50% of all menopausal women. The symptoms are caused by the lack of oestrogen and they result in atrophy of the epithelium in both vagina and urethra. The symptoms include dryness, discomfort, pain, recurrent urinary tract infections, urge incontinence and stress incontinence (frequent urinations and urinations during night time). The problem could be overseen in the group of catheter users, because of their primary bladder dysfunction, impaired sensation and basic incontinence.

Thinning of the urethral mucosa can increase risk of urethral trauma and thereby increase risk of urinary tract infections. The hormonal changes also affects pH to increase, which contributes to the risk of urinary tract infections. Furthermore, thinning of the urethral mucosa can cause lack of urethral pressure and thus stress incontinence.

Estrogen replacement therapy is often used to control menopause related urinary incontinence, and this treatment is based on the fact, that some estrogens in high degree stimulate the estrogen receptors in the urethral and bladder wall. By stimulating the estrogen receptors locally, the mucosa lining will increase in thickness and restore urethral pressure, pH and thereby help control urinary incontinence.

The Urogenital Oestrogen Deficiency Syndrome in itself is often solved by treating with systemic or vaginal administration of oestrogen or oestrogen-derivatives, such as Oestriol or Oestradiol.

It is known that Oestriol or Oestradiol treatment increases the mobility of the urethro-vesical junction (Martan A. et al. Ceska Gynekol. 1999 January; 64(1):6-9), increases the thickness of urethral mucosa (Henriksson L. et al., Am J Obstet Gynecol. 1994 September; 171(3):624-32), increases urethral vascularisation (Martan A. et al. Ceska Gynekol. 1999 January; 64(1):6-9), alleviates subjective and objective symptoms (Henriksson L. et al., Am J Obstet Gynecol. 1994 September; 171(3):624-32), restores vaginal pH (Henriksson L. et al., Am J Obstet Gynecol. 1994 September; 171(3):624-32) and decreases leakage episodes and urinary incontinence complaints (Ahistom K. et al., Gynecol Obstet Invest. 1990; 30(1):37-43). The results are, however, varying depending on the administration route, but it can be concluded that vaginal administration bypasses the first liver metabolism and is therefore more potent and shows better results and lesser side effects (Heimer GM. Estriol in the menopause. Acta Obstet Gynecol Scand Suppl 1987; 139:1-23).

Agents for treating overactive bladder are used in patients with idiopathic overactive bladder or neurogenic bladder disorders, for instance caused by Spinal Cord Injury. The patients usually have severe detrusor hyperreflexia plus a disorder of bladder emptying, and intermittent catheterisation is the preferred method of bladder management to minimise residual urine. Catheterisation is often accompanied by treatment to increase bladder capacity and reduce bladder spasms.

Strategies for treating over active bladder can be to lessen the parasympathetic activity (parasympatolytica) or increasing sympathetic activity locally. Another principle is blocking the afferent arm of the bladder contraction reflex.

Blocking the parasympathetic activity (block of pelvic nerve-detrusor smooth muscle colinergic transmission) with parasympatolytica is the most widely used treatment principle, using anticholinergic agents, such as oxybutynin, tolterodine etc. This treatment principle is based on blocking the efferent pathway in the bladder contraction reflex arch. When given systemically, parasympatolytica also affects other organs, such as the mouth, eyes and bowel system. Parasympatolytica in general can have unpleasant side effects on other systems, e.g. dry mouth, accomodation difficulties, tendency of constipation. Parasympatolytica with specific effect on the bladder is therefore preferable. Furthermore, it is shown that intravesical (local) administration of anticholinergic drugs has very good effect and produces fewer side effects than orally administered anticholinergics.

Blocking the afferent arm of the bladder contraction reflex involves blocking of the nerve-pathways from the musculature of the bladder to the spinal cord. This group of drugs include Capsaicin (a chilli peber extract) Resiniferatoxin (RTX) and Local anaesthetic drugs.

In summary medical treatment accompanying catheterisations may be beneficial for a number of catheter users. However, systemic administration of a pharmaceutically active composition, often requires a higher dose of active agents, have more side-effects and is often not as efficient as a local treatment. On the other hand local treatment of the urinary tract system is often performed as a procedure with the only objective of medical treatment, i.e. for catheter users, in addition to the procedure of catheterisation, another procedure must be performed to receive the local medical treatment. It is an object of the present invention to overcome these disadvantages by providing a device for urinary catheterisation, which combines urinary catheterisation with a local medical treatment, in that agents for the medical treatment is delivered by the catheter element during the usual catheterisation process.

### SUMMARY OF THE INVENTION

A first aspect of the invention relates to a device for urinary catheterisation, said device comprising a catheter element adapted to be inserted in the urethra of a human, and a pharmaceutically active composition comprising at least one agent selected from the group consisting of hormones, efferent blocking agents, afferent blocking agents and sympathomimetic agents, and said catheter element is adapted to deliver at least a part of said pharmaceutically active composition in the lower urinary tract system during catheterisation.

A second aspect of the invention relates to the use of a pharmaceutically active composition comprising at least one agent selected from the group consisting of hormones, efferent blocking agents, afferent blocking agents and sympathomimetic agents, for the manufacture of a device for the treatment, alleviation or prophylaxis of incontinence in a human, said device comprising a catheter element adapted to be inserted in the urethra of said human, said catheter element comprising the pharmaceutically active composition, and said catheter element being adapted to deliver said agent in the lower urinary tract system during catheterisation.

A third aspect of the invention relates to a method of treating a human suffering from or being susceptible to incontinence, the method comprising the steps of catheterisation of said human by arranging a proximal end of a catheter element of a device for urinary catheterisation in the urethra of said human, said catheter element comprising a pharmaceutically active composition comprising at least one agent selected from the group consisting of hormones, efferent blocking agents, afferent blocking agents and sympathomimetic agents, and said catheter element being adapted to deliver said composition in the lower urinary tract system during catheterisation.

A forth aspect of the invention relates to a kit comprising a device for urinary catheterisation and a pharmaceutically active composition comprising at least one agent selected from the group consisting of hormones, efferent blocking agents, afferent blocking agents and sympathomimetic agents, said device comprising a catheter element adapted to be inserted in the urethra of a human.

A fifth aspect of the invention relates to a device for urinary catheterisation, said device comprising a catheter element adapted to be inserted in a urinary canal, said device further comprising a discrete unit dose, said discrete unit dose comprising a pharmaceutically active composition and said catheter element being adapted to shed said pharmaceutically active composition in the lower urinary tract system during catheterisation.

### BRIEF DESCRIPTION OF THE DRAWINGS

The invention is disclosed more in detail with reference to the drawings in which

FIG. 1-4 shows examples of patterns formed by zones of active ingredients (1) on a tubular catheter element (12) with eyes (9).

FIG. 5 shows a package with a compartment (5) containing a gel. The compartment has a tip (2), which is removed before use and a sealing (3) which is broken as the catheter element (4) is pushed through the compartment containing the gel (1).

FIG. 6 shows a cross section (length direction) of a catheter element (6) without eyes and with a discrete unit dose in the shape of a pill (7) placed on the proximal end of the catheter element. The discrete unit dose further comprises a film (8) covering the pill.

FIG. 7 shows a cross section (perpendicular to the length direction) of a wing catheter (10) with a substance containing active ingredients in the concave corner (11).

### DETAILED DESCRIPTION OF THE PRESENT INVENTION

Device for Urinary Catheterisation

This invention relates to a device for urinary catheterisation, said device comprising a catheter element adapted to be inserted in the urethra of a human, and a pharmaceutically active composition comprising at least one agent selected from the group consisting of hormones, efferent blocking agents, afferent blocking agents and sympathomimetic agents, and said catheter element is adapted to deliver at least a part of said pharmaceutically active composition in the lower urinary tract system during catheterisation.

A device according to the invention comprises a pharmaceutically active composition, which has an effect on the continence system, and a catheter element for drainage of urine from the bladder and for delivering the pharmaceutically active composition in the urethra during the catheterisation procedure. In addition to catheterisation, a device according to the invention is thus performing a local medical treatment of the lower urinary tract system, such as urethra or bladder with the aim of treatment, alleviation or prophylaxis of incontinence.

Urinary catheterisation may be performed using an intermittent or an indwelling catheter. In a preferred embodiment of the invention, a device is provided for intermittent catheterisation. The proximal end of the catheter element is adapted to be inserted in the urethra, typically the catheter element is part of a urinary catheter, the urinary catheter further comprising a handle or connecter element attached to the distal end of the catheter element. For most embodiments, the type of catheter element is not essential to the invention and could be of any type, such as disclosed in PCT/DK02/00449. In addition to tubular catheters for which the drainage canal is defined by the catheter material, preferable embodiments of the present invention could comprise a catheter element of wing catheter type, for which the urethra form a part of the drainage canals. The catheter element may be adapted to fit the urethra or males, females or children.

The delivery of the pharmaceutically active composition in the lower urinary tract system may include active delivery mechanisms, such as injection of the active agents through a cavity in the catheter, which may be separated from the cavity used for drainage of urine. Such active delivery mechanisms typically complicates the device and its use, as the pharmaceutically active composition is often provided separately and additional steps in the procedure of catheterisation is introduced due to the delivery of the pharmaceutically active composition.

More preferred is a passive delivery mechanism, i.e. the catheter element carries the pharmaceutically active composition on the outside surface of the insertable part of the catheter element, i.e. the surface adapted to contact the urethra. At least a part of the pharmaceutically active composition is deposited in the urinary tract system during catheterisation as a result of the friction, body heat, humid environment, osmosis etc. encountered in the body. Preferably the pharmaceutically active composition is delivered in the urinary tract system solely by such passive mechanism, eliminating the need for additional steps in the procedure of catheterisation due to the delivery of a pharmaceutically active composition. This also simplifies the device, as the pharmaceutically active composition is typically an integrated part of the catheter element.

The part of the pharmaceutically active composition to be delivered in the urinary tract system may be placed on the outer surface of a proximal part of the catheter element prior to insertion, in accordance with a passive delivery mechanism, as the outer surface is brought in contact with the cavities of the urinary tract system, so that the pharmaceutically active composition can be passively delivered from the catheter element.

In a preferred embodiment of the invention, a major part of said pharmaceutically active composition is present on an outer surface of the catheter element before insertion of said catheter element. Typically substantially all of the pharmaceutically active composition may be present on an outer surface of the catheter element prior to insertion of the catheter element.

In a further embodiment of the invention the device is provided in a sealed package, wherein a major part of said pharmaceutically active composition is present on an outer surface of the catheter element, i.e. the catheter element is pretreated with the pharmaceutically active composition prior to opening the package to expose said catheter element.

In one embodiment of the invention the catheter element is adapted for intermittent catheterisation. In this case the catheter element should be able to deliver the pharmaceutically active composition very quickly, i.e. in less than 2 minutes, which is the average normal time for intermittent catheterisation. The active ingredient should preferably work in the urethra for approx. 2-4 hours, in between catheterisations. In another embodiment of the invention the active substance is released more slowly and the time spend by the catheter element in the urethra by intermittent catheterisation is extended. In a further embodiment the release of the active agents is adapted to take place with the catheter element permanently placed in the urethra.

In one embodiment of the invention, the catheter element is comprised in a female catheter. Accordingly the catheter element is adapted to fit the female urethra, i.e. it has a length in the range of 50-200 mm, such as 130-180 mm, such as in a length in the size of 150 mm or even as short as 50-90 mm, such as in the range of 55-85 mm, such as in the range of 60-80 mm, such as a length in the size of 70 mm.

In one embodiment of the invention, at least a part of the active composition is provided in a coating covering at least a portion of the outer surface of a proximal part of the catheter element and the coating is adapted to release said pharmaceutically active composition within the lower urinary tract system. The coating could be a polymer coating, of which at least a portion is impregnated with at least a part of the pharmaceutically active composition. At least a portion of the catheter could have a hydrophilic coating adapted to reduce friction between the catheter element and urethra for a more comfortable insertion. In one embodiment of the present invention this hydrophilic coating and/or the swelling medium for swelling the hydrophilic coating may contain at least a part of the pharmaceutically active composition. In another embodiment of the present invention a hydrophilic coating and a coating containing the active ingredients could be applied to the catheter element in an alternating pattern to create zones adapted to deliver active ingredients alternating with zones adapted to reduce friction. Examples of patterns of distribution are shown in FIG. 14. The zones of active ingredients could be constrained to a part of the catheter element such as the tip. The coating containing active ingredients could have hydrophobic properties, e.g. for resistance to a liquid swelling medium.

In one embodiment of the invention the pharmaceutically active composition is distributed over a section of the catheter element having a length of at least 50% of the total length of the catheter element. A section of the catheter element is a part of the catheter element bounded by one or two cross sections perpendicular to the long axis of the catheter element. This drug delivering section may constitute a major section of the catheter element having a length of at least 50%, such as at least 60%, such as at least 70%, such as at least 80%, such as at least 90% of the total length of the catheter element, such as essentially the full insertable length of the catheter element. Thus the treatment may be extended to a major part of the urethra.

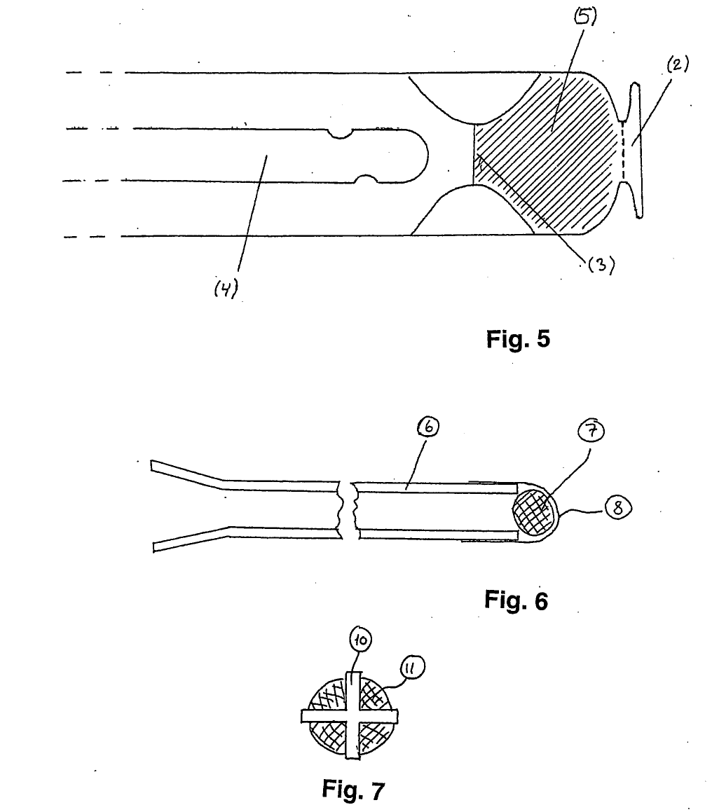

Also a high-viscosity coating may be provided by means of a gel or crème or alternatively liquid, solution or spray may be used. In one embodiment at least a part of the active composition is provided in a gel or crème adapted for application to at least a portion of the catheter element. In a preferred embodiment the active composition is integrated in a gel that pre-lubricates the catheter element to reduce friction between the catheter element and urethra for a more comfortable insertion. In this case there is no need for a hydrophilic coating, since the gel in itself would provide the lubricating effect. In one embodiment of the present invention the gel is applied in the production procedure, i.e. the catheter is provided in a pre-treated condition. In another embodiment the gel and the catheter element is provided in a catheter assemblage adapted for application of the gel to the catheter element prior to use by the person performing the catheterisation. An example is a catheter package including an Oestriol-containing gel. In an embodiment of the invention the gel is provided in a separate container adapted for application of the gel to the catheter element by squeezing the container. In another embodiment the package hosting the catheter element has a compartment adapted to hold the gel. In a further embodiment the gel is applied to the catheter element by pressing the catheter element through the compartment containing the gel. An example of this solution is shown in FIG. 5.

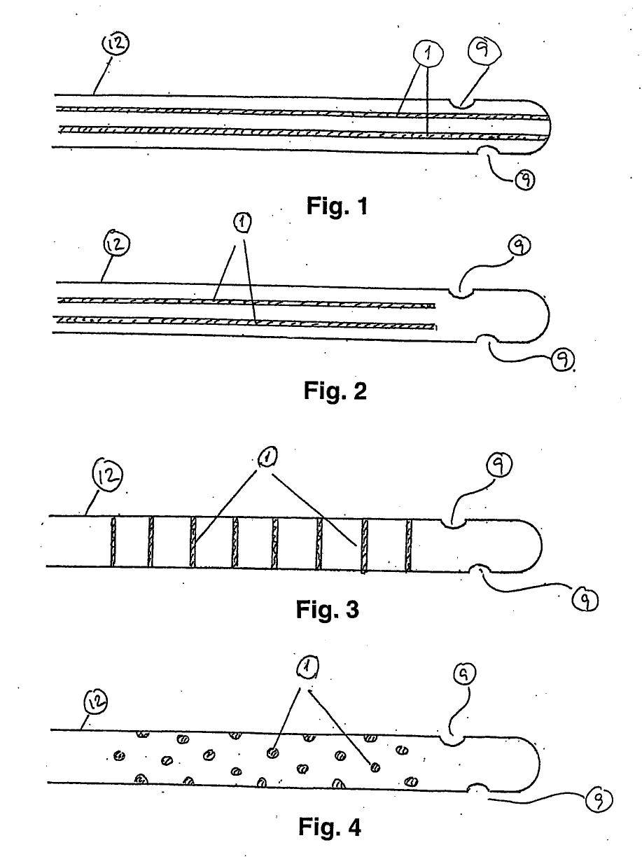

In another embodiment the catheter element has depressions on the outer surface, which are adapted to hold at least a part of the active agents. In case of a wing catheter active ingredients could be provided in the concave corners of the catheter as shown in FIG. 7. FIG. 1-4 gives examples of a tubular catheter with depressions forming a pattern of zones containing at least one active ingredient. In a further embodiment of the invention, release of the active ingredients in the urethra is promoted by a consistency regulating agent, which e.g. become more viscous when warmed to body temperature, and is either used in the matrix or as a slip layer between catheter element and the pharmaceutical composition or as a cover on top of the active ingredients which is melted or dissolved by contact with urine and/or body heat.

The catheter element may be provided with a capping covering at least a part of the pharmaceutically active composition. Preferably the capping comprises a material, which is dissolved or melted by contact with urine and/or body heat, e.g. PVA.

In another embodiment of the invention, at least a part of the pharmaceutically active composition is provided in a separate discrete unit dose, such as a pill or ampoule, and the catheter device is adapted to insert this discrete unit dose in the lower urinary tract system. In one embodiment a pill could be placed on the tip of the catheter and capped with a film, which is dissolved or melted by contact with urine and/or body heat, such as PVA. An example of this embodiment is shown in FIG. 6. This solution has the advantage that a tubular catheter element does not need eyes, i.e. drainage holes in the side of the tubular member, since the pill provide a rounded end of the catheter element for comfortable insertion. Hence the catheter can be made about 2 cm shorter and discomfort due to the eyes is avoided.

A further aspect of the invention is to provide a kit comprising a device for urinary catheterisation and a pharmaceutically active composition comprising at least one agent selected from the group consisting of hormones, efferent blocking agents, afferent blocking agents and sympathomimetic agents, said device comprising a catheter element adapted to be inserted in the urethra of a human.

In particular the catheter element and the pharmaceutically active composition may be provided separately. This may in particular be the case when the pharmaceutically active composition in provided in the form of a gel or creme for application to the catheter element.

Pharmaceutically Active Composition

The present invention allows for medical treatment of the continence system.

In a first embodiment of the invention, the pharmaceutically active composition contains active agents for treatment of the urethral mucosa, such as agents effective against urethral atrophy. Certain hormones have proven valuable for preventing and treating urethral atrophy. Examples include oestrogens and oestrogen derivatives such as oestriol or oestradiol.

In one embodiment of the invention the pharmaceutically active composition comprises at least one hormone. In a further embodiment of the invention the hormone is a female sex hormone. This embodiment of the device is especially suitable for treating incontinence in women, e.g. by treatment with a pharmaceutically active composition effective against urethral atrophy. Also in women with imperative urge to urinate and possibly accompanying urge incontinence because of postmenopausal changes in the urethral mucosa, estrogens (e.g. estradiol and estriol) have a positive effect on these symptoms.

In a further embodiment of the invention the hormone is estrogen or an estrogen derivative. Estrogens may increase urethral pressure by increasing the thickness of the urethral mucosa. Moreover, estrogens may increase the number of adrenergic receptors on urethral smooth muscle. The estrogen can have several different forms including natural, synthetic, or semi-synthetic compounds. Examples of estrogens include estradiol, diethyl stilbestrol, estrone, estrone sodium sulfate, sodium equilin sulfate, ethinyl estradiol, quinestrol, diethylstilbestriol, mestranol, estriol, and chlorotrianisene. In a preferred embodiment of the invention the pharmaceutically active composition comprises oestriol or oestradiol.

By coating an intermittent catheter with a hormone such as Oestriol or Oestradiol, the active ingredient is delivered to the urethral mucosa directly and this treatment could alleviate the symptoms caused by urethral atrophy in between the catheterisations.

The primary effect of the device is drainage of urine, the secondary effect is supplying the urethral epithelium with oestriol to achieve better continence in between catheterisations by mucosal proliferation and additional effects as described above.

Compared to known treatments, the purpose of urethral administration is to achieve better effect on urological symptoms than vaginal or oral administration, and to avoid the side effects seen by systemic administration.

In another embodiment of the present invention at least a part of the pharmaceutically active composition could be selected to have an effect on the unstriated musculature or the neuromuscular junction. Examples of such active ingredients with a desired effect comprise anticholinergical drugs and capsaicin.

The pharmaceutically active composition may comprise efferent blocking agents for lessening the parasympathetic efferent activity (blocking the pelvic nervedetrusor smooth muscle colinergic transmission), and/or agents for increasing sympathetic activity and/or afferent blocking agents for blocking the afferent arm of the reflex causing the bladder contraction may also be used.

Efferent blocking agents includes parasympatolytica or spasmolytica, such as anticholinergica. Examples are Cetiprin (Emepron), Detrusitol (Tolterodin), Urispadol (Flavoxat), Atropine, oxybutynin and Spasmo-Lyt (Trospiumchlorid).

In one embodiment of the invention the pharmaceutically active composition comprises an efferent blocking agent selected from the group consisting of anticholinergical agents, sympathomimetics agents, alfa-adrenergic agonists and nicotinic cholinergic agonists.

In a preferred embodiment of the invention the pharmaceutically active composition comprises oxybutynin or trospiumchlorid.

In another embodiment of the invention the pharmaceutically active composition comprises an afferent blocking agent, such as Capsaicin, RTX and Local anaesthetic drugs.

When treating detrusor hyperreflexia, the group of parasympatolytica or spasmolyticalytica can be used to relax the bladder wall musculature. Parasympatolytica in general can have unpleasant sideeffects on other systems, e.g. dry mouth, accomodation difficulties, tendency of constipation. Parasympatolytica with specific effect on the bladder is therefore preferable. It is shown that intravesical (local) administration of anticholinergic drugs has very good effect and produces fewer sideeffects than orally administrered anticholinergics.

The pharmaceutically active composition may also comprise sympathomimetic agents (agents increasing sympathetic activity). Sympathomimetic agents generate urethral pressure by increasing the tone of the internal sphincter. The sympathomimetic agent will stimulate the .alpha.-adrenergic receptors in the internal sphincter, which will increase its tone. The internal sphincter will then tighten around the urethra and the neck of the bladder.

alpha-Adrenergic agonists are one type of sympathomimetic agent that can be effective. Various types of alpha-adrenergic agents include phenylephrine HCl, pseudoephedrine HCl, phenylpropanolamine HCl, ephedrine sulfate, norephedrine HCl, xylometazoline HCl, oxymetazoline HCl, naphazoline HCl, norepinephrine HCl, and privine HCl. Examples of other sympathomimetic agents include norepinephrine uptake inhibitors such as desipramine HCl, amitriptyline HCl, desmethylimipramine HCl, and imipramine HCl. Yet another sympathomimetic agent includes norepinephrine releasing agents such as tyramine.

Nicotinic cholinergic agonists and acetylcholinesterase inhibitors increase the tone of the external sphincter. Additionally, either of these types of agents can be combined with muscarinic cholinergic antagonist such as atropine, scopolamine, or glycopyrrolate. In this type of treatment, the agent will stimulate the nicotinic cholinergic receptors in the external sphincter, which will increase its tone and cause it to tighten around the urethra. Examples of nicotinic cholinergic agonists include choline, acetylcholine, methacholine, carbachol, bethanechol, arecoline, and 1,1-dimethyl-4-phenylpiperazinium iodide. Examples of acetylcholinesterase inhibitors include physostigmine salicylate, neostigmine bromide, ambenomium chloride, edrophonium chloride, demecarium bromide, and pyridostigmine bromide.

Additionally, estrogens and sympathomimetics such as an alpha-adrenergic agonist can be used in combination. Current medical research indicates that estrogens may increase the number of alpha-adrenergic receptors in the internal sphincter. Thus, the alpha-adrenergic agonists will stimulate both the preexisting and newly developed alpha-adrenergic receptors. The increased number of alpha-adrenergic receptors will cause the sphincter muscles to respond more efficiently to the alpha-adrenergic agonists and have an even greater increase in tone.

The pharmaceutically active composition may in addition to the therapeutic agents for medical treatment of incontinence also comprise enhancing agents for enhanced penetration of the therapeutic agents through the urothelium lining of the urethra and into the tissue of the urethral wall. Examples of penetration enhancers include 1->2-(decylthio)ethyl!azacyclopentan-2-one; 1-dodecylazacycloheptan-2-one; dimethylsulfoxide; 1-menthol; and 1-lauryl-2-pyrrolidone.

Medical Treatment

A further aspect of the invention relates to the use of a pharmaceutically active composition comprising at least one agent selected from the group consisting of hormones, efferent blocking agents, afferent blocking agents and sympathomimetic agents, for the manufacture of a device for the treatment, alleviation or prophylaxis of incontinence in a human, said device comprising a catheter element adapted to be inserted in the urethra of said human, said catheter element comprising the pharmaceutically active composition, and said catheter element being adapted to deliver said agent in the lower urinary tract system during catheterisation.

The catheter element comprises the pharmaceutically active composition, in the form of an impregnation, coating, substance distributed over the catheter or in any other way previously disclosed. Also the device may have any features previously described.

In one embodiment of the invention the use in particular relates to females.

A further aspect of the invention is to provide a method of treating a human suffering from or being susceptible to incontinence, the method comprising the steps of catheterisation of said human by arranging a proximal end of a catheter element of a device for urinary catheterisation in the urethra of said human, said catheter element comprising a pharmaceutically active composition comprising at least one agent selected from the group consisting of hormones, efferent blocking agents, afferent blocking agents and sympathomimetic agents, and said catheter element being adapted to deliver said composition in the lower urinary tract system during catheterisation.

The catheter element comprises the pharmaceutically active composition, in the form of an impregnation, coating, substance distributed over the catheter or in any other way previously disclosed. Also the device may have any features previously described.

In one embodiment of the invention the method in particular relates to females.

Device Comprising Discrete Unit Dose

A second objective of the invention is to provide a device for urinary catheterisation, comprising a catheter element with a proximal end adapted for insertion in a urinary canal, and a discrete unit dose containing a pharmaceutically active composition. The catheter element is adapted to shed the discrete unit dose in the lower urinary tract system during catheterisation.

The urinary canal is in particular the natural urethra of men, women or children, but the invention may in some cases be useful even for delivering a pharmaceutically active composition through an artificial urinary canal to the bladder. The catheter element may be e.g. a tubular catheter for draining urine through an internal duct or a wing catheter for draining urine in external ducts partly bounded by the urinary canal, the catheters being provided in dimensions to fit the urinary canals in males, females or children.

The pharmaceutically active composition may comprise any active agents suitable for administration in the lower urinary tract system e.g. antibacterial agents, or active agents for treatment of incontinence, such as disclosed in this document regarding the first aspect of the invention.

The discrete unit dose may be a pill, tablet, capsule, ampoule etc, containing a pharmaceutically active element comprising a pharmaceutically active composition. The pharmaceutically active element may be in solid form, shaped to constitute the discrete unit dose. The discrete unit dose may also comprise a capping, in the shape of a film or coating, covering at least a part of the pharmaceutical active element. This is e.g. advantageous when the pharmaceutically active element is not in solid form. The discrete unit dose may be attached to the catheter by means of the capping. Preferably the capping is made of a material, which is dissolved or melted by contact with urine and/or body heat, e.g. PVA. In this manner the discrete unit dose is released by the insertion into the urinary tract system. In one embodiment of the invention the pharmaceutically active element is placed in contact with the catheter element and a capping, such as a film covers the discrete unit dose and a proximal part of the catheter element. In another embodiment the capping extends at least between the catheter element and the discrete unit dose. In this case the discrete unit dose may adhere to the catheter element by means of the capping material. As an alternative the discrete unit dose may be unattached to the catheter element. According to this alternative the catheter element may have a depression, wherein the discrete unit dose may be seated. As an example the catheter element may have a depression at the tip, wherein the discrete unit dose may be seated, to be pushed through the urinary in front of the catheter element.

The pharmaceutically active element may be deposited in the urethra during catheterisation, e.g. during insertion of the catheter. Typically, however, the pharmaceutically active element composition is delivered in the bladder.

In a preferred embodiment of the invention the discrete unit dose is placed on the tip of the catheter. Usually, urinary catheters have a rounded tip, to allow comfortable insertion of the catheter and avoid damaging urethra and bladder. The opening or openings in a tubular catheter for drainage of urine are thus placed as ‘eyes’ in the side wall a small distance, e.g. in the order of one or a few cm from the tip. In a preferred embodiment of the present invention, the catheter has a tubular proximal section, the proximal end of said tubular proximal section having an opening for draining urine from the outside of the catheter to the inside of the tubular section. The discrete unit dose may then be placed proximally at the tip of the catheter element, to provide a smooth tip thereby preventing the edges of the opening from cutting in the urinary canal. Evidently the unit dose may also be used in combination with a wing catheter to provide a smooth tip on the catheter element. For a tubular catheter element a further advantage is that the catheter element does not need eyes, i.e. drainage openings in the side of the tubular member, since a drainage opening is provided in the proximal end of the tubular section, with the discrete unit dose providing a rounded, smooth tip on the catheter, to support comfortable and non-traumatic insertion of the catheter. Hence the catheter can be made about 2 cm shorter. Furthermore the eyes typically present in known catheters must be carefully rounded and smoothed to prevent cutting in the urethra and even then the eyes may still cause discomfort and damage to the tissue, as the urethra in some cases tends to be sucked into the eyes of the catheter, especially during withdrawal of the catheter from the bladder.

In one embodiment the drainage openings of the catheter are covered by the discrete unit dose and the drainage openings are uncovered as the discrete unit dose is delivered in the body of a human.

1. A device for urinary catheterisation comprising a catheter element adapted to be inserted in the urethra of a human, said catheter element comprising on the outer surface, before insertion of the catheter element, a pharmaceutically active composition comprising at least one agent selected from the group consisting of hormones, efferent blocking agents, afferent blocking agents and sympathomimetic agents, such that the pharmaceutically active composition is delivered to the lower urinary tract system during catheterisation.

2. A device according to claim 1, wherein said pharmaceutically active composition comprises a hormone.

3. A device according to claim 1, said device being provided in a sealed package, wherein a major part of said pharmaceutically active composition is present on an outer surface of the catheter element.

4. A device according to claim 1, wherein the pharmaceutically active composition is distributed over a section of the catheter element having a length of at least 50% of the total length of the catheter element.

5. A device according to claim 1, wherein the catheter element is adapted for intermittent catheterisation.

6. A device according to claim 1, wherein said catheter element is comprised in a female catheter.

7. A device according to claim 1, wherein said catheter element has a coating covering at least a portion of the outer surface of the catheter element and said coating contains at least a part of said pharmaceutically active composition and is adapted to release said pharmaceutically active composition within the lower urinary tract system.

8. A device according to claim 1, wherein at least a part of said catheter element has a polymer coating, and at least a portion of said polymer coating is impregnated with at least a part of said pharmaceutically active composition.

9. A device according to claim 1, wherein at least a portion of said catheter element has a hydrophilic coating.

10. A device according to claim 9, wherein said hydrophilic coating is impregnated with at least a part of said pharmaceutically active composition.

11. A device according to claim 1, wherein said catheter element has depressions on the outer surface, which are adapted to hold at least a part of said pharmaceutically active composition.

12. A device according to claim 1, wherein at least a part of said pharmaceutically active composition is provided in a gel or creme.

13. A device according to claim 1, wherein said device is comprising a lubricating gel adapted to reduce friction between the catheter element and urethra, and said gel is containing at least a part of said pharmaceutically active composition.

14. A device according to claim 1, wherein said device is comprising a discrete unit dose containing said pharmaceutically active composition said device is adapted to shed said discrete unit dose in the lower urinary tract system.

15. A device according to claim 1, wherein said hormone is a female sex hormone or a derivative thereof.

16. A device according to claim 15, wherein said hormone is selected from oestrogen or an oestrogen derivative.

17. A device according to claim 15, wherein said hormone is oestriol or oestradiol.

18. A device according to claim 1, wherein said pharmaceutically active composition comprises an efferent blocking agent selected from the group consisting of anti-cholinergical agents, sympathomimetics agents, alfa-adrenergic agonists and nicotinic cholinergic agonists.

19. A device according to claim 19, wherein said efferent agent is oxybutynin or trospiumchlorid.

20. A device according to claim 1, wherein said pharmaceutically active composition comprises an afferent blocking agent.

21. Use of a pharmaceutically active composition comprising at least one agent selected from the group consisting of hormones, efferent blocking agents, afferent blocking agents and sympathomimetic agents, for the manufacture of a device for the treatment, alleviation or prophylaxis of incontinence in a human, said device comprising a catheter element adapted to be inserted in the urethra of said human, said catheter element comprising the pharmaceutically active composition, and said catheter element being adapted to deliver said agent in the lower urinary tract system during catheterisation.

22. The use according to claim 21, wherein the human is a female.

23. The use according to claim 21, wherein the device is as defined in a device for urinary catheterisation comprising a catheter element adapted to be inserted in the urethra of a human, said catheter element comprising on the outer surface, before insertion of the catheter element, a pharmaceutically active composition comprising at least one agent selected from the group consisting of hormones, efferent blocking agents, afferent blocking agents and sympathomimetic agents, such that the pharmaceutically active composition is delivered to the lower urinary tract system during catheterisation.

24. A method of treating a human suffering from or being susceptible to incontinence, the method comprising the steps of catheterisation of said human by arranging a proximal end of a catheter element of a device for urinary catheterisation in the urethra of said human, said catheter element comprising a pharmaceutically active composition comprising at least one agent selected from the group consisting of hormones, efferent blocking agents, afferent blocking agents and sympathomimetic agents, and said catheter element being adapted to deliver said composition in the lower urinary tract system during catheterisation.

25. The method according to claim 24, wherein the human is a female.

26. The method according to claim 23, wherein the device is as defined in a device for urinary catheterisation comprising a catheter element adapted to be inserted in the urethra of a human, said catheter element comprising on the outer surface, before insertion of the catheter element, a pharmaceutically active composition comprising at least one agent selected from the group consisting of hormones, efferent blocking agents, afferent blocking agents and sympathomimetic agents, such that the pharmaceutically active composition is delivered to the lower urinary tract system during catheterisation.

27. A kit comprising a device for urinary catheterisation and a pharmaceutically active composition comprising at least one agent selected from the group consisting of hormones, efferent blocking agents, afferent blocking agents and sympathomimetic agents, said device comprising a catheter element adapted to be inserted in the urethra of a human.

28. A device for urinary catheterisation, said device comprising a catheter element with a proximal end adapted to be inserted in a urinary canal, characterised in that said device is comprising a discrete unit dose, said discrete unit dose comprising a pharmaceutically active composition and said catheter element being adapted to shed said pharmaceutically active composition in the lower urinary tract system during catheterisation.

29. A device according to claim 28, wherein said discrete unit dose is placed at the tip of the catheter.
